(19) United States
(12) Patent Application Publication
(10) Pub. No.: US 2007/0031281 A1
(43) Pub. Date: Feb. 8, 2007

(54) Oven with ultraviolet sterilizer

(76) Inventor: Carl M. Stevens, Aurora, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/196,770

(22) Filed: Aug. 2, 2005

(51) Int. Cl. A61L 2/10; A61L 2/12

(52) U.S. Cl. 422/24; 422/21

(57) ABSTRACT

An oven system, including apparatus and methods, for use in sterilizing solid and/or liquid materials. The oven may include a (1) cavity, (2) a heating mechanism for heating objects within the cavity, (3) a UV lamp for sterilizing objects within the cavity, and/or (4) a controller for allowing users selectively to engage and control the heating mechanism and/or the UV lamp.

### BACKGROUND

Hygiene-related illnesses such as food poisoning afflict tens of millions of people a year in the United States alone. These illnesses can cause considerable discomfort or death, and cost billions of dollars a year in medical care and lost productivity. Commercial products commonly are treated to reduce or prevent hygiene-related illness. For example, food products may be treated by pasteurization, irradiation, chemical treatment, and so on. However, in some cases, these treatments may alter the flavor and/or nutritional content of the food. In other cases, improper post-commercial handling of food may contaminate or re-contaminate otherwise sterile food. In all cases, the common commercial treatments may be expensive and/or dangerous. Thus, there is a need for a consumer or end-user level device for sterilizing objects such as foodstuffs and hygiene-related products, particularly a device that is safe and easy to operate, and that may be used if desired without heating or cooking.

### SUMMARY

The present teachings provide an oven system, including apparatus and methods, for use in sterilizing solid and/or liquid materials. The oven may include a (1) cavity, (2) a heating mechanism for heating objects within the cavity, (3) an ultraviolet (“UV”) lamp for sterilizing objects within the cavity, and/or (4) a controller for allowing users selectively to engage and control the heating mechanism and/or the UV lamp.

### BRIEF DESCRIPTION OF THE DRAWINGS

FIG. 1 shows a box diagram of an exemplary oven system, for heating and/or sterilizing objects, in accordance with aspects of the present teachings.

FIG. 2 shows a side view of an embodiment of the oven of FIG. 1. This embodiment employs a conventional (e.g., electric or gas) heating element.

FIG. 3 shows a side view of another embodiment of the oven of FIG. 1. This embodiment employs a microwave heating element.

FIG. 4 shows a box diagram of a circuit from the oven of FIGS. 1-3.

FIG. 5 shows a box diagram of an alternative circuit from the oven of FIGS. 1-3.

### DETAILED DESCRIPTION

The present teachings provide an oven system, including apparatus and methods, for use in sterilizing solid and/or liquid materials. The oven may include (1) a cavity, (2) a heating mechanism for heating objects within the cavity, (3) an ultraviolet (“UV”) lamp for sterilizing objects within the cavity (e.g., by irradiating them with UV light), and/or (4) a controller for allowing users selectively to engage and control the heating mechanism and/or the UV lamp.

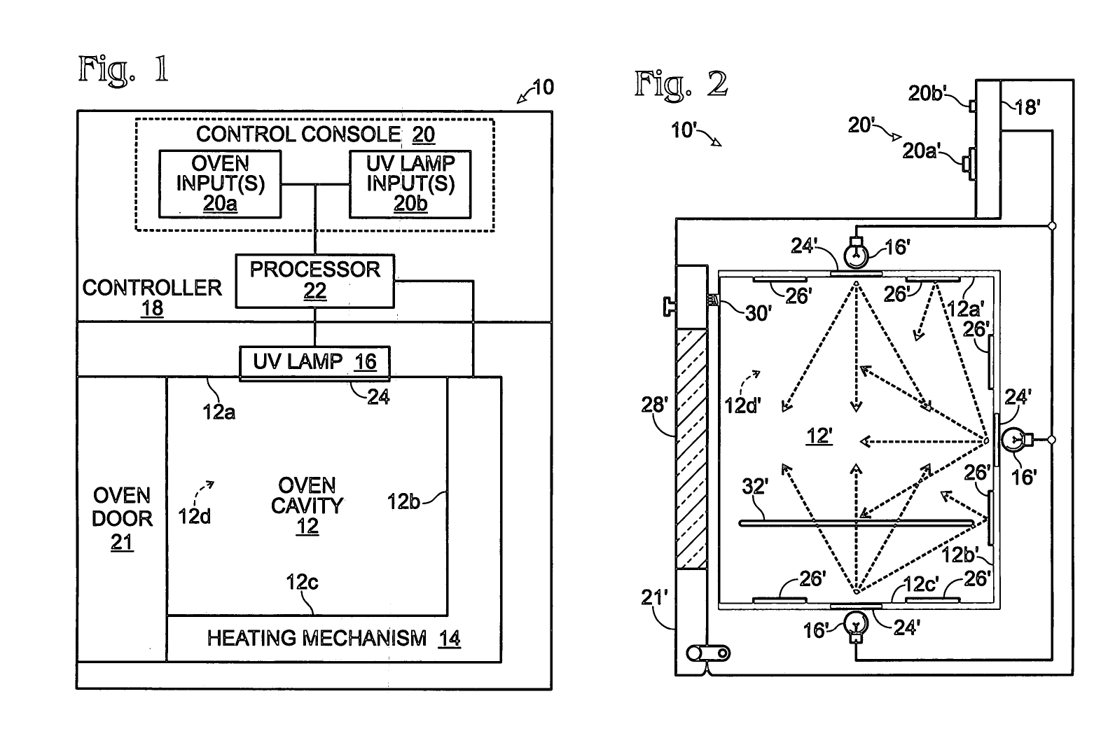

FIG. 1 shows a box diagram of an exemplary oven system 10, in accordance with aspects of the present teachings. The oven system may include a cavity 12, a heating mechanism 14, a UV lamp 16, and/or a controller 18. The cavity may be configured to retain objects of various shapes and sizes for heating and/or UV sterilization. The heating mechanism may be positioned adjacent to or within the cavity, and may be configured to heat objects retained in the cavity. The UV lamp also may be positioned adjacent to or within the cavity, and may be configured to produce sterilizing UV light within the cavity. The controller may include oven input(s) 20a for selectively engaging and operating the heating mechanism, and UV lamp input(s) 20b for selectively engaging and operating the UV lamp. These inputs may be independent of, and/or coupled to, one another.

UV light, such as that used in the oven system, is invisible electromagnetic radiation with wavelengths typically ranging between about 100 and 400 nanometers (“nm”). This light may be divided further into several, arbitrary wavelength regimes, including (1) UV-C (from about 100 nm to about 280 or 290 nm), (2) UV-B (from about 280 or 290 nm to about 315 or 320 nm), and (3) UV-C (from about 315 or 320 nm to about 400 nm). Shorter-wavelength, higher-energy UV light (particularly UV-C light) can be used to kill or disable (e.g., render nonreproducing and/or noninfectious) biological contaminants, such as bacteria, mold/fungi, and viruses, among others. UV light tends to be absorbed or otherwise dissipated by both solids and liquids (particularly opaque liquids); therefore, UV light is best used to sterilize the surfaces of solid objects and either translucent, or thin films of, liquid objects.

The oven system, and components thereof, may be manufactured from any suitable material(s), including, but not limited to, metals (such as stainless steel, aluminum alloys, etc.), plastic, rubber, glass, quartz, and/or ceramic, among others, including combinations thereof. These materials may be selected and/or finished to satisfy any suitable criteria, including strength, durability, appearance, and ease of cleaning and use, among others. For example, as discussed below, the walls defining the cavity may be made of material(s) that conduct heat and/or reflect microwave radiation and/or UV light, among others.

The following sections further describe further aspects and embodiments of the present teachings, including (I) the cavity, (II) the heating mechanism, (III) the UV lamp, (IV) the controller, and (V) examples, among others.

I. The Cavity

FIGS. 1-3 show exemplary aspects of an oven cavity, in accordance with aspects of the present teachings. The cavity generally comprises any mechanism for retaining objects of various shapes and sizes for heating and/or UV sterilization. The cavity may include one or more components, and have any suitable size and shape, consistent with its function(s). Exemplary cavities are described below, including (a) a schematic oven cavity, (b) an exemplary conventional oven cavity, and (c) an exemplary microwave oven cavity.

I.a Schematic Oven Cavity

The cavity may be defined by any suitable combination of walls, doors, and/or windows, among others. For example, a typical cavity might have five walls and a door, defining a six-sided (e.g., rectangular) enclosure, and a suitable although optional combination of internal and/or external windows disposed in the walls and/or doors. Specifically, a typical cavity may be defined by a top wall 12a, a rear wall 12b, a bottom wall 12c, two side walls 12d, and a door 21. The walls and/or door(s) may include (1) material(s) that conduct or insulate from heat, (2) material(s) that reflect thermal and/or microwave radiation and/or UV light, among others, and/or (3) holes for ventilating and/or circulating air within the cavity. The walls also may include an internal window 24 made, at least in part, of a translucent material, such as quartz or any other non-UV-absorbing material, for allowing UV light to pass into the cavity from a UV lamp 16 positioned adjacent to the cavity. The door(s) (and in some cases the walls) also may include an external window made, at least in part, of a translucent material that permits a user to look from the outside to the inside of the cavity. The door(s) 21 may be movable between an open position and a closed position. When the door is open, objects may be placed into and/or removed from the cavity. When the door is closed, the door may partially or fully define a wall such as a front wall of the cavity, thereby preventing heat, microwave radiation, and/or UV light from escaping the cavity. The door also may include a securing mechanism, such as a latch, a seal, and/or a biasing mechanism, among others, for securing the door in a closed position.

I.b Exemplary Conventional Oven Cavity

FIG. 2 shows an exemplary conventional (or convection) oven 10′, such as an electric, gas, or convection oven, among others, in accordance with aspects of the present teachings. In this example, a cavity 12′ may be defined by five walls 12a′-d′ and a door 21′ made of heat conducting material(s), and/or materials that reflect UV light, among others. For example, the walls may include at least one reflector 26′, such as a mirror or other light-reflective material, for reflecting UV light within the cavity. The at least one reflector may be configured relative to the at least one UV lamp such that UV radiation is evenly distributed to most or substantially all (pertinent) areas within the cavity. The door may include an external window 28′ made, at least in part, of a translucent material that permits a user to look from the outside to the inside of the cavity. The external window also may be made of a material that reduces or prevents the emission of heat and/or UV light to the outside of the cavity. For example, the windows may be made of glass of sufficient thickness to absorb and/or reflect most or all of the UV light produced within the cavity by the UV lamp. The external window thereby may protect someone looking through the window from the deleterious effects of UV radiation. As an additional safety precaution, the oven ‘further may include a door switch 30’ that automatically disengages the UV lamp and/or the heating mechanism when the door is opened, as discussed below.

The cavity further may include a support 32′ for holding objects. The support may be made of metal and/or other material(s), that is heat conductive, heat resistant, durable, lightweight, and/or easy to clean, among others. For example, the support may be a typical oven rack that engages slots in side walls 12d′, and supports objects in the center portion of the cavity to permit uniform heating of the objects. The support also may include a rack for holding specific objects, such as containers, foods (e.g., fruits, eggs, etc.), food-preparation products (e.g., cutting boards, utensils, etc.), hygiene-related products (e.g., razors, clippers, scissors, thermometers, files, etc.), and so on. The containers may include any receptacle such as bottles or jars suitable for holding foods, drinks, hygiene-related products, and/or any other suitable object. Finally, the support may include a rotisserie or carousel for rotating or reorienting objects within the cavity.

I.c Exemplary Microwave Oven Cavity

FIG. 3 shows an exemplary microwave oven 10″, in accordance with aspects of the present teachings. In this example, the cavity 12″ may be defined by five walls 12a-d″ and a door 21″ made of heat conducting material(s), and/or materials that reflect microwave radiation and/or UV light, among others. For example, the walls may include at least one reflector 26″ for reflecting UV light within the cavity. The reflector may be selected so that it is nonreactive to microwave radiation. The reflector also may be selected so that it reflects or channels microwave radiation. Further, the reflector may be configured relative to the at least one UV lamp such that UV radiation is evenly distributed to most or substantially all (pertinent) areas within the cavity. The door may include an external window 28″ made, at least in part, of a translucent material that permits a user to look from the outside to the inside of the cavity, while reducing or preventing the emission of UV light from the inside to the outside of the cavity, as described above. The window also may include a screen or other mechanism that reduces or prevents the emission of microwave radiation to the outside of the cavity. As an additional safety precaution, the oven may include a door switch 30″ that automatically disengages the UV lamp and the heating mechanism when the door is opened, as discussed below.

The cavity 12″ of microwave oven 10″ further may include a support 32″ for holding objects. The support may be made of glass, plastic, crystal, pyrex, or any other material, that is nonreactive to microwave radiation, durable, lightweight, and/or easy to clean, among others. The support may include a rack for holding specific objects, and/or a rotisserie or carousel for rotating or reorienting objects within the cavity, as described above for conventional oven cavities.

II. The Heating Mechanism

FIGS. 1-3 show exemplary oven heating mechanisms, in accordance with aspects of the present teachings. The heating mechanism, as indicated above, generally comprises any mechanism(s) for heating objects within the cavity. The heating mechanism may include one or more components, and may have any suitable size and shape, consistent with its function(s). Moreover, the heating mechanism may be combined with other, similar or dissimilar heating mechanism(s). The heating mechanism(s) may be positioned adjacent to and/or within the cavity, depending on the type of oven. The heating mechanism(s) may include conventional (e.g., electric and/or gas) mechanism(s), convection mechanism(s), and/or microwave mechanism(s), among others.

Conventional ovens (e.g., as shown in FIG. 2), such as electric or gas ovens, include heating mechanisms that heat the air within the cavity, which in turn heats objects within the cavity. Electric ovens typically have a heating mechanism that includes at least one electric heating element positioned within and above the bottom wall of the cavity (e.g., for baking and roasting), and/or within and below the top wall of the cavity (e.g., for broiling). These electric heating elements may include resistors that heat the air inside the cavity when an electric current is passed through the resistor. Gas ovens typically have a heating mechanism that includes at least one gas burner positioned adjacent to and below the bottom wall of the cavity (e.g., for baking and roasting), and/or adjacent to and above the top wall of the cavity (e.g., for broiling). Each burner may be connected to a gas valve, and an optional igniter, such as a spark generator or pilot light, may be positioned adjacent to the burner. When the gas valve is opened, inflammable gas is emitted from the burner, and the igniter ignites the gas, thereby heating the air surrounding the flame. The cavity walls adjacent to the burner may include holes that permit the heated air to enter the cavity as it expands.

Convection ovens typically have a heating mechanism that heats air in the same manner as either an electric oven or a gas oven, as discussed above. However, in addition, the heating mechanism of convection ovens further includes a fan for circulating the heated air within the cavity, thereby increasing the efficiency of the oven by increasing the efficiency of heat transfer between the air and objects in the oven.

Microwave ovens (e.g., as shown in FIG. 3) typically have a heating mechanism 14″ that includes a magnetron 34″ for producing microwaves positioned adjacent to the cavity, and a wave guide 36″ for containing the microwaves and guiding them into the cavity. In contrast to conventional ovens, the heating mechanism of microwave ovens does not heat the air within the oven's cavity (although heated objects may incidentally heat the air). Rather, microwaves produced by the magnetron cause polar molecules, such as water and fat molecules in food, to vibrate, thereby generating heat. To ensure uniform heating of polar molecules within the cavity, and therefore even cooking of food within the cavity, the heating mechanism of microwave ovens also may include some form of dispersal mechanism adapted to evenly disperse microwaves within the cavity. For example, the dispersal mechanism may include a fan 38″ positioned within or adjacent to the cavity and along the path of the microwaves. The fan may be adapted to reflect the microwaves in varying directions so that the microwaves are dispersed substantially evenly throughout (pertinent portions of) the cavity. Alternatively, or in addition, microwave ovens may include a support 32″ that functions as a carousel. The carousel may rotate objects within the cavity to ensure that most of the polar molecules within the object are evenly exposed to and excited by microwave radiation.

Combination ovens may include two or more different heating mechanisms, for example, (1) conventional and convection, (2) conventional and microwave, (3) convection and microwave, or (4) conventional, convection, and microwave, among others.

III. The UV Lamp

FIGS. 1-3 show exemplary UV lamps, in accordance with aspects of the present teachings. The UV lamp, as indicated above, generally comprises any mechanism(s) for producing sterilizing UV light within the cavity. The UV lamp may include one or more components, and may have any suitable size and shape, consistent with its function(s). The UV lamp may be positioned adjacent to and/or within the cavity for producing UV light within the cavity.

The UV lamp preferably produces effective amounts of sterilizing UV light. Here, sterilizing (or sanitizing) may include destroying contaminants, reducing the number and/or effectiveness of contaminants (e.g., to levels judged safe by public health standards), and/or disabling contaminants (e.g., preventing viable reproduction and/or growth of biological contaminants), among others. For example, sterilization may include killing and/or inactivating living biological contaminants, such as bacteria, mold/fungi, and/or the like. Alternatively, or in addition, sterilization may include inactivating nonliving biological contaminants, such as viruses, virions, prions, and/or the like. Here, “inactivate” means to render an agent that otherwise would be capable of causing infection, illness, and/or the like either less effective or ineffective at causing such effects. Sterilization may involve any suitable mechanism(s), such as death, DNA dimer formation, denaturation, cleavage, and/or the like.

UV-C light, as discussed above, (particularly UV-C light with a wavelength of about 245 nm), may be especially efficacious for sterilization. Thus, in some embodiments, at least about a quarter or a third or a half of the light emitted by the UV lamp may be UV-C light. Highly intense UV lamps (also known as germicidal lamps) are available commercially, in a number of suitable configurations.

The time required for sterilization may depend on a number of factors, including the intensity and spectrum of UV light emitted by the lamp, the relative placements and orientations of the lamp and object, the nature of the object being sterilized, the nature of the contaminants being treated, and so on. For example, UV light with greater intensity generally sterilizes objects in relatively shorter times, whereas UV light with lesser intensity generally sterilizes objects in relatively longer times. Moreover, solids and liquids absorb UV radiation as a function of depth of penetration and their extinction coefficient. Therefore, depending on the type of object that is being decontaminated, the relative positions of contaminants relative to the surface, the intensity of the UV light produced by a selected UV lamp, and so on, relatively shorter or longer exposure times may be necessary. In some embodiments, more than one UV lamp may be included in the oven to ensure that the UV light is sufficiently intense, and/or that it is produced from various directions relative to an object placed within the cavity. Alternatively, or in addition, focusing and/or reflective elements may be included in the oven to help intensify and/or direct UV light, respectively.

IV. The Controller

FIGS. 1-3 show controllers 18, 18′ for various ovens 10. The controller, as indicated above, generally comprises any mechanism(s) capable of engaging and controlling the heating mechanism and UV lamp. The controller may have (1) a first input device for selectively engaging the heating mechanism, and (2) a second input device for selectively engaging the UV lamp. The controller may include one or more components, and have any suitable size and shape, consistent with its function(s). These components may include manual switches and/or electronic (e.g., software) switches.

The controller may include a control console 20 for selectively engaging the heating mechanism (14) and/or the UV lamp (16) (see, e.g., FIG. 1). The control console may include buttons, toggles, switches, dials, timers, and/or any other devices for controlling the operation of an oven. For example, the control console may include one or more oven inputs 20a that allows a user selectively to engage the heating mechanism, and one or more UV lamp inputs 20b that allows a user selectively to engage the UV lamp.

The controller also may include a processor 22 for processing user input(s) and appropriately and selectively engaging the heating mechanism and/or the UV lamp.

The controller 18′ for a conventional oven may include a control console 20′ for selectively engaging the heating mechanism and the UV lamp (16′) (see, e.g., FIG. 2). The control console may include oven input(s) 20a′ that allow a user selectively to engage the heating mechanism, and a UV lamp input 20b′ that allows a user selectively to engage the UV lamp. The oven input(s) (20a′) may include buttons, toggles, dials, and/or other devices for controlling the temperature of the air within the cavity of a conventional oven, and/or the heating/cooking time of the oven. The UV lamp input(s) (20b′) may include buttons, toggles, dials, and/or other devices for turning the UV lamp on or off, and/or for selecting the intensity and/or duration of the UV light produced by the UV lamp.

The controller for a microwave oven may include a control console 20″, and a processor 22″ for selectively engaging the magnetron (34″) and the UV lamp (16″) (see, e.g., FIG. 3). The control console 20″ may include oven input(s) 20a″ that allow a user selectively to engage the magnetron, and a UV lamp input 20b″ that allows a user selectively to engage the UV lamp. The oven input(s) (20a″) may include buttons, toggles, dials, and/or other devices for controlling the power of microwaves produced by the magnetron, and/or the heating/cooking time of the oven. The UV lamp input (20b″) may include buttons, toggles, dials, and/or other devices for turning the UV lamp on or off, and/or for selecting the intensity and/or duration of the UV light produced by the UV lamp. User input signals are processed by processor 22″, which may include circuitry for engaging the magnetron and the UV lamp. The processor also may include a transformer, rectifier, magnetic field circuit and other microwave oven components for generating the necessary power for the magnetron to produce high energy microwave radiation.

The controller, including control console and processor, for other ovens, such as convection and/or combination ovens, may include similar combinations of inputs and abilities. These may allow selection between alternative heating elements (e.g., conventional and microwave), fans (in the case of ovens involving convective elements), and so on.

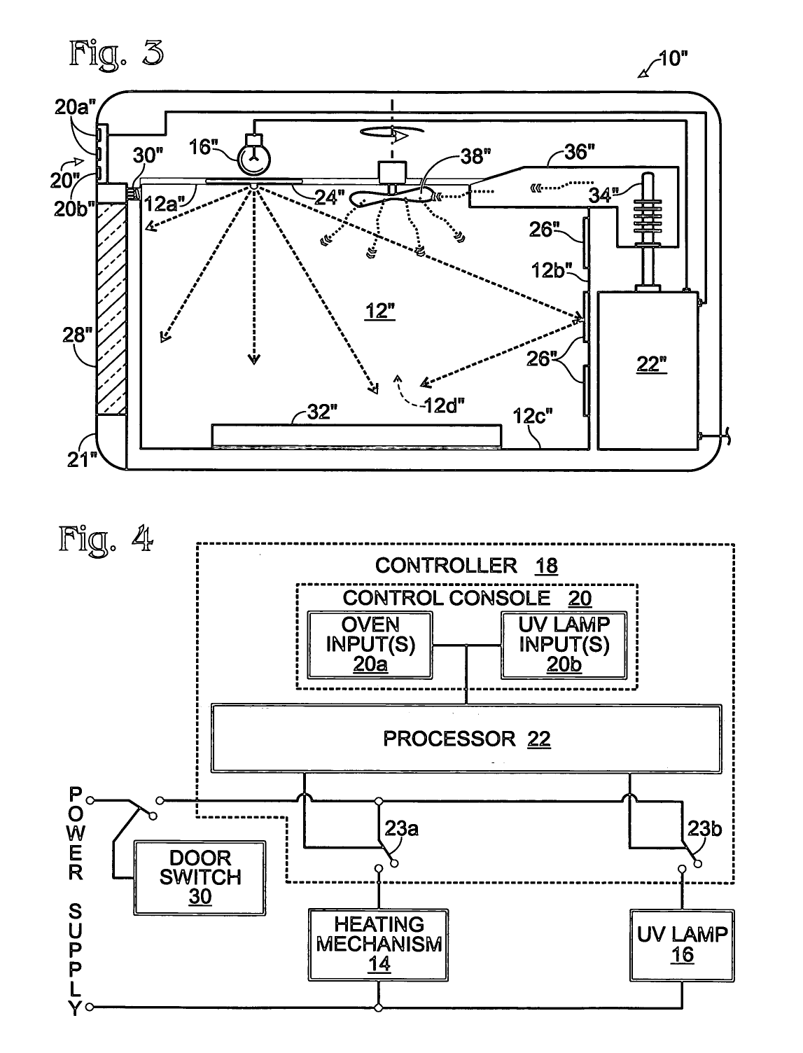

The controller may be configured to allow a user independently to engage either the heating mechanism and/or the UV lamp (e.g., as shown in FIG. 4). Specifically, the controller may include a switch 23a that is actuated by the processor 22 when a user actuates the oven input(s), thereby engaging the heating mechanism regardless of the operational state of the UV lamp. The controller also may include a switch 23b that is actuated by the processor when a user actuates the UV lamp input, thereby engaging the UV lamp regardless of the operational state of the heating mechanism. In such a configuration, a user may choose to use the UV lamp to sterilize objects, without heating/cooking the objects. A user also may choose to use the UV lamp in conjunction with the heating mechanism, such as before, during, and/or after heating/cooking the objects.

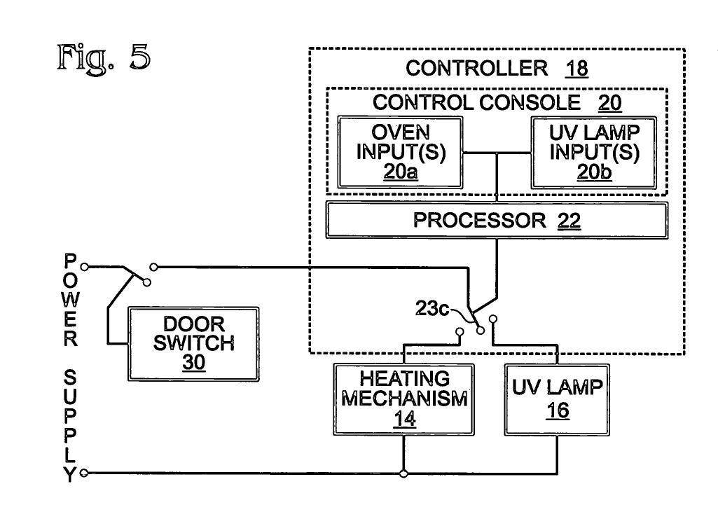

Alternatively, the controller may be configured to prevent a user from engaging the UV lamp and the heating mechanism simultaneously (e.g., as shown in FIG. 5). The controller may include a switch 23c that is actuated by processor 22 when a user actuates either the oven input(s) (20a) or the UV lamp input (20b), thereby engaging either the heating mechanism (14) or the UV lamp (16), but not both at the same time. For example, engagement of the UV lamp may be trumped by engagement of the heating mechanism. Specifically, if a user attempts to engage both the heating mechanism and the UV lamp, the processor may be configured only to engage the heating mechanism, and to disengage the UV lamp. However, if the heating mechanism is manually turned off, or an oven timer automatically shuts off the heating mechanism, the processor may be configured to re-engage the UV lamp.

The oven 10 also may include a door switch 30 that automatically disengages the UV lamp and/or the heating mechanism when the door is opened. For example, the door switch may be configured automatically to disengage both the UV lamp and the heating mechanism when the door is opened, as shown in FIGS. 4 and 5. Thus, for a microwave oven, the door switch may act as a safety mechanism that prevents harmful UV light and/or microwaves from escaping the oven's cavity. Alternatively, the door switch may be configured to disengage only the UV lamp when the door is opened. Thus, for conventional and/or convection ovens, in particular, the door switch may act as a safety mechanism that prevents harmful UV light from escaping the oven's cavity, without interrupting heating.

### EXAMPLES

The ovens discussed above may allow a user to sterilize objects, such as foods, drinks, containers, and/or hygiene-related products, in an oven without heating and/or cooking the objects. For example, a user may sterilize the surface of a solid object, or a translucent, or thin film of, a liquid object, by placing the object inside the cavity of the oven, engaging the UV lamp with the UV lamp input, and irradiating the object with UV light. This may be especially useful for sterilizing plastics (and/or other materials) that would melt when heated in a conventional oven and/or metal (and/or other materials) that would react or interfere with microwaves in a microwave oven. This also may be useful for sanitizing or decontaminating food that otherwise would change flavor and/or lose nutritional value when heated or cooked, such as juices, raw eggs, milk, cheese, and so on. The sterilized object thereafter may be removed from the oven, and a second object may be placed inside the oven for heating with the heating mechanism and/or sterilization with the UV lamp.

The ovens also may allow a user also to choose to use UV light in conjunction with the heating mechanism of a conventional or microwave oven, such as before and/or after heating/cooking objects. For example, a user first may use the heating mechanism to cook food in an oven. Then, after the user has finished cooking the food, the user may desire to allow the food to sit and cool inside the oven before serving or storing the food. To prevent biological organisms from contaminating the surface of the food, the user may engage the UV lamp until the food is removed from the oven and served, and/or stored in a refrigerator, freezer, or other food storage device.

The disclosure set forth herein may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. An oven, comprising:
a cavity;
a heating mechanism for heating objects within the cavity;
a UV lamp for producing UV light within the cavity; and
a controller having a first input device for selectively engaging the heating mechanism, and a second input device for selectively engaging the UV lamp.

2. The oven of claim 1, wherein the UV lamp produces sterilizing UV light.

3. The oven of claim 2, wherein at least about half of the light emitted by the UV lamp is UV-C light.

4. The oven of claim 1, further comprising a door with a transparent window that selectively transmit visible light to permit a user to look inside the cavity, while selectively reducing or preventing the transmission of UV light to the outside of the cavity.

5. The oven of claim 1, further comprising a door, and a switch that automatically disengages the UV lamp when the door is opened.

6. The oven of claim 1, wherein the cavity includes at least one reflector for reflecting UV light within the cavity.

7. The oven of claim 1, wherein the cavity includes a support for holding objects.

8. The oven of claim 7, wherein the support includes a rack configured to hold a preselected object.

9. The oven of claim 7, wherein the support includes a carousel for rotating objects within the cavity.

10. The oven of claim 1, wherein the oven is a microwave oven, and the heating mechanism includes a magnetron for producing microwaves.

11. The microwave oven of claim 10, wherein the controller is configured to engage the heating mechanism upon user actuation of the first input device, and the UV lamp upon user actuation of the second input device.

12. The microwave oven of claim 11, wherein the controller is configured to prevent a user from engaging the heating mechanism and the UV lamp simultaneously.

13. The oven of claim 1, wherein the heating mechanism heats the air within the cavity.

14. The oven of claim 13, wherein the oven is a gas oven, and the heating mechanism includes a burner, a gas valve connected to the burner, and an igniter positioned adjacent to the burner.

15. The oven of claim 13, wherein the oven is an electric oven, and the heating mechanism includes an electric heating element.

16. The electric oven of claim 15, wherein the controller is configured to engage the heating mechanism upon user actuation of the first input device, and the UV lamp upon user actuation of the second input device.

17. The electric oven of claim 16, wherein the controller is configured to prevent a user from engaging both the heating mechanism and the UV lamp simultaneously.

18. The oven of claim 13, wherein the oven is a convection oven, and the heating mechanism includes a fan for dispersing heated air within the cavity.

19. A method of sterilizing living biological contaminants on the surface of objects, the method comprising:
placing a first object inside a cavity of an oven comprising:
a heating mechanism for heating objects within the cavity;
a UV lamp for producing UV light within the cavity; and
a controller having a first input device for selectively engaging the heating mechanism, and a second input device for selectively engaging the UV lamp;
engaging the UV lamp with the second user input device; and
irradiating the object with UV light.

20. The method of sterilizing living biological contaminants on the surface of objects of claim 19, further comprising:
removing the first object from the cavity;
placing a second object in the cavity; and
engaging either the heating mechanism or the UV lamp.
